(19) United States
(12) Patent Application Publication
(10) Pub. No.: US 2011/0082226 A1
(43) Pub. Date: Apr. 7, 2011

(54) MULTI-ISOCYANATE PREPOLYMER

(75) Inventors: Fan Jeng Tsai, Hsinchu City (TW); Yih-Her Chang, Hsinchu City (TW); Chih-Chien Chen, Hsinchu City (TW); Yuung-Ching Sheen, Jhubei City (TW); Kai-Wen Chang, Taichung City (TW)

(73) Assignee: INDUSTRIAL TECHNOLOGY RESEARCH INSTITUTE, Hsinchu County (TW)

(21) Appl. No.: 12/650,496

(22) Filed: Dec. 30, 2009

(30) Foreign Application Priority Data
Oct. 1, 2009 (TW) 098133347

(51) Int. Cl. C08G 18/32; C07D 251/30

(52) U.S. Cl. 521/170; 544/222

(57) ABSTRACT

Disclosed is a multi-isocyanate prepolymer formed by reacting diisocyanate and/or diisocyanate trimer with diol and/or triol. Because the prepolymer and the diol/triol have similar molecular weights, additional crosslink agents such as diamine, multi-amine, or combinations thereof are not necessary. Furthermore, the prepolymer can be blown to form foam, or directly gelled to form an unbubbled product such as film.

### CROSS REFERENCE TO RELATED APPLICATIONS

This Application claims priority of Taiwan Patent Application No. 98133347, filed on Oct. 1, 2009, the entirety of which is incorporated by reference herein.

### BACKGROUND

1. Technical Field

The disclosure relates to foam, and in particular relates to a non-yellowing foam prepolymer and formula and method for manufacturing the same.

2. Description of the Related Art

Polyurethane (PU) is an important material used in essential goods and industrial material and is widely applied in the transportation, sports, furniture, packaging, clothing, and thermal insulation material industries, and the likes. In North America, Europe and Japan, PU production is greater than 2.3 million tons per year, and its global growth rate is 2% to 3% per year. PU foam factories in Taiwan produce almost 100 thousand tons per year.

While the PU industry develops with time goes by, the modern foam products have developed several technologies such as novel blowing agent, non-yellowing assistant, stabilizer, force-cool forming, variation pressure blowing, carbon dioxide system, and isophorone diisocyanate (IPDI) series PU foam and related process. The current focus in PU development tends to environmental friendly (halogen-free materials), non-yellowing, and high quality.

Aliphatic diisocyanates such as IPDI or hexamethylene diisocyanate (HDI), do not contain aromatic rings. Thus, aliphatic diisocyanates do not degrade (e.g. yellow) easily. Accordingly, aliphatic diisocyanates are applied in blowing clothes such as underwear, hygiene instruments, traffic instruments, buffer materials, and the likes. The global value of aliphatic diisocyanates is about NT$ 3 bn to 3.5 bn dollars.

The isocyanate has a functional group —NCO with high reactivity. Isocyanates mostly react with nucleophile reactants as shown in Formulae 1-3.

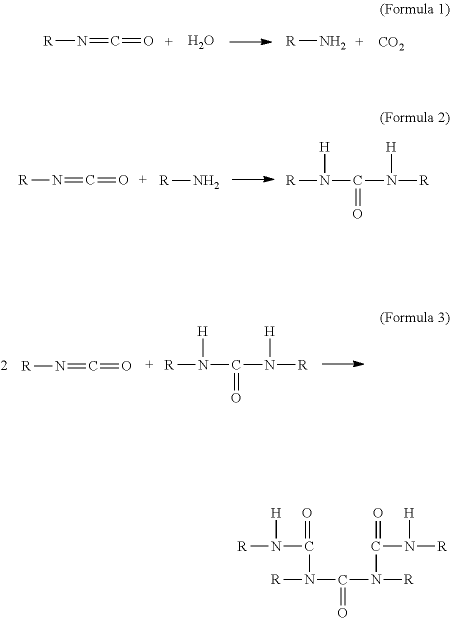

Formula 1 shows the isocyanate reacting with water to form amine, Formula 2 shows the isocyanate reacting with the amine in Formula 1 to form urea by amidation, and Formula 3 shows the reaction of isocyanate with the urea in Formula 2. R is a saturated alkyl group. In practice, Formulae 1-3 are continuous reactions which occur before setting.

The aliphatic isocyanate such as IPDI are yellowing resistant; however, its reactivity is lower than the aromatic isocyanate. If the prepolymer is selected to be foam precursor, the reactivity of isocyanate at two terminals will be further decreased due to increasing molecular weight of the prepolymer. Therefore, a novel prepolymer formula is developed for overcoming the poor PU properties due to insufficient reactivity of the diisocyanate prepolymer.

### SUMMARY

Disclosed embodiments of the invention provide a multi-isocyanate prepolymer obtained by reacting a diisocyanate trimer with a diol, wherein the diisocyanate trimer and the diol have a molar ratio from 2:1 to 4:1.

The embodiments also provide a multi-isocyanate prepolymer obtained by reacting a diisocyanate prepolymer with a triol, wherein the diisocyanate prepolymer and the triol have a molar ratio from 3:1 to 6:1. The diisocyanate prepolymer is obtained by reacting a diisocyanate with a diol, wherein the diisocyanate and the diol have molar ratio from 2:1 to 4:1.

A detailed description is given in the following embodiments with reference to the accompanying drawings.

### DETAILED DESCRIPTION OF DISCLOSED EMBODIMENTS

The following description is of the best-contemplated mode of carrying out the invention. This description is made for the purpose of illustrating the general principles of the invention and should not be taken in a limiting sense. The scope of the invention is best determined by reference to the appended claims.

As shown in Formula 5, the embodiment provides a prepolymer obtained by reacting a diisocyanate trimer as shown in Formula 4 with a diol.

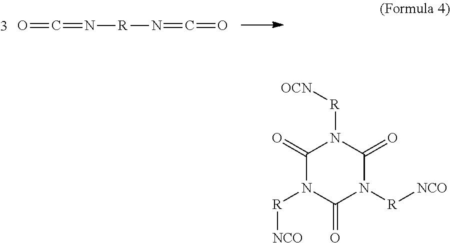

To prepare the diisocyanate trimer, a suitable diisocyanate includes aliphatic isocyanate such as isophorone diisocyanate (IPDI), hexamethylene diisocyanate (HDI), methylene-bis(4-cyclohexylisocyanate), 4,4′-dicyclohexylmethane diisocyanate (H₁₂MDI), and the likes. The diisocyanate trimer can be synthesized according to J. S. Ferguson (Toxicology and applied pharmacology, page 332-346 (1987)), N3600 or N3400 commercially available from Bayer (Germany), or Basonat I commercially available from BASF (Germany). In one embodiment, the diol is a polymer having two hydroxyl groups at two terminals, respectively. The diol, having molecule weight from 200 to 3000, can be polyester polyol or polyether polyol such as polyethylene glycol (PEG), polypropylene glycol (PPG), and the likes.

As shown in Formula 5, the diol is mixed with the diisocyanate trimer to react therewith for forming the prepolymer. Note that the molar ratio of the diisocyanate trimer and the diol should be greater than 2:1, preferably 2:1 to 4:1. If the diisocyanate trimer and the diol have a molar ratio less than 2:1 (e.g. 1:1), the mixture tends to form long-chained polymer as shown in Formula 6 rather than the prepolymer as shown in Formula 5.

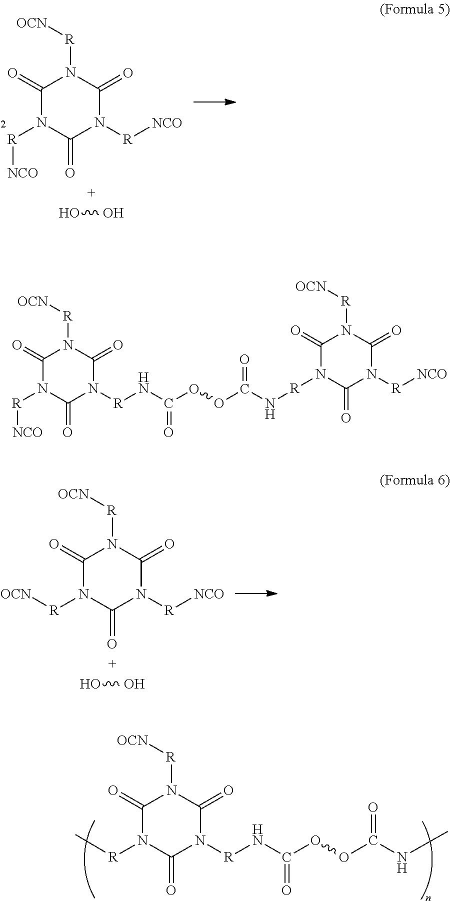

In another embodiment, the prepolymer formula may further include diisocyanate to obtain a prepolymer having 2 to 4 diisocyanate functional groups, as shown in Formula 7. The diisocyanate includes IPDI, HDI, methylene-bis(4-cyclohexylisocyanate), H₁₂MDI, or combinations thereof. The further added diisocyanate and the diisocyanate monomer of the trimer can be the same (R═R′) or different (R≈R′). The diisocyanate and the diisocyanate trimer can be added to the reactor simultaneously or separately. As described above, the total molar of the diisocyanate and the diisocyanate trimer should be twice of the molar of the diol, such that the product will be the prepolymer other than the long-chained polymer.

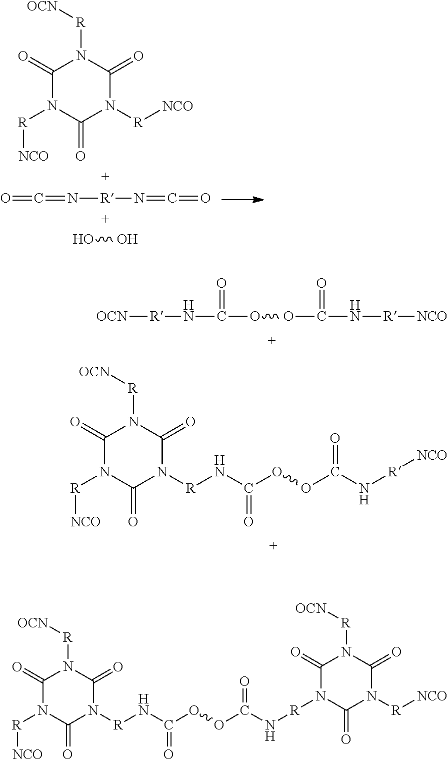

In a further embodiment, the multi-isocyanate prepolymer is obtained by a two-step process. Firstly, the diisocyanate prepolymer is prepared by reacting the diol and the diisocyanate, as shown in Formula 8. In Formula 8, the diisocyanate and the diol have a molar ratio greater than 2:1, preferably 2:1 to 4:1, to form prepolymer other than the long-chained polymer. Subsequently, the diisocyanate prepolymer in Formula 8 and the triol react to form a triisocyanate prepolymer, as shown in Formula 9. In Formula 9, the diisocyanate prepolymer and the triol have a molar ratio greater than 3:1, preferably 3:1 to 6:1, to form the prepolymer other than the long-chained polymer. In one embodiment, the triol is a polymer having three hydroxyl groups at three terminals, respectively. The triol, having molecule weight from 200 to 3000, can be polyester polyol or polyether polyol such as polyethylene glycol (PEG), polypropylene glycol (PPG), and the likes. Note that the diisocyanate prepolymer in Formula 9 cannot be replaced with other multi-isocyanates having more than three isocyanate groups. If the triol is reacted with the multi-isocyanate, it will form a polymer other than the prepolymer. It should be emphasized that the diisocyanate prepolymer and the triol have similar molecular weights, such that the terminal hydroxyl groups of the triol can be completely reacted. If the general diisocyanate such as HDI is adopted to replace the diisocyanate prepolymer in Formula 8, the product in Formula 9 contains some hydroxyl groups and does not completely reacted to form the diisocyanate groups. An incomplete reaction of the triol and the HDI is caused by the molecular difference between the triol and the HDI. As such, any remaining hydroxyl groups of the prepolymer molecules will react with the isocyanate groups of prepolymer molecules, and result in the storage time of the prepolymer to be dramatically decreased, such as less than 3 months.

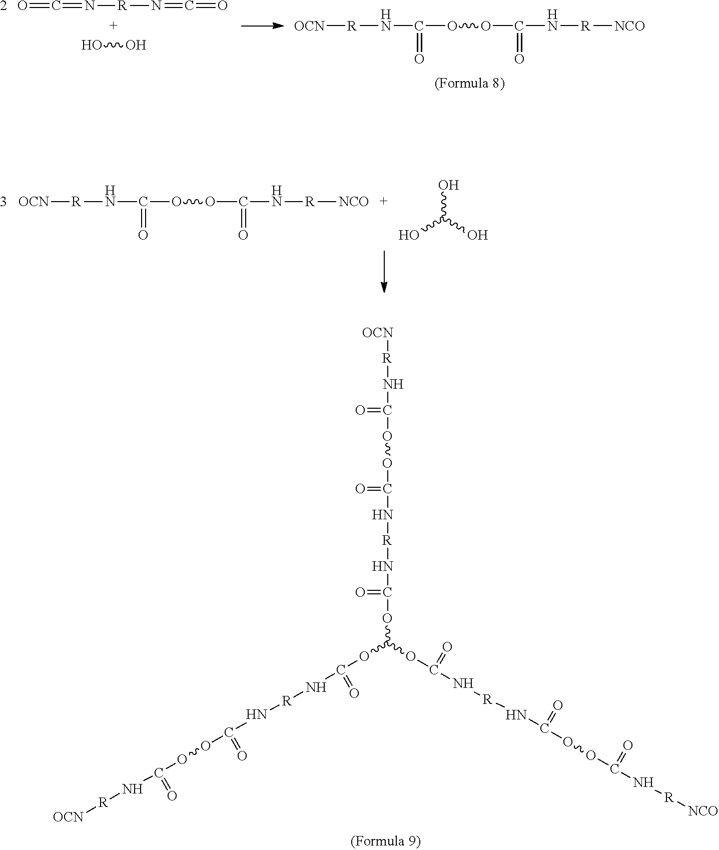

The prepolymer such as the products of Formulae 5, 7, or 9, surfactant, catalyst, blowing agent, and water are mixed to perform blowing and gelling reaction. At last, the blown polymer is solidified to form foam. Compared with the related art, the prepolymers of the embodiment are obtained by reacting the diisocyanate trimer with the diol or obtained by reacting the diisocyanate prepolymer with the triol, such that the prepolymers of the embodiment have 4 terminal reactive groups (Formula 5), 2 to 4 terminal reactive groups (Formula 7), or 3 terminal reactive groups (Formula 9). Accordingly, poor properties of conventional aliphatic isocyanate prepolymers due to the low reactivity are improved. Furthermore, the prepolymer of the embodiment may be easily and quickly blown and gelled at room temperature.

In one embodiment, the prepolymer and the water have a preferably weight ratio of 100:0.1 to 100:10. Note that too much water will let the foam crack, and too little water will let the forming ratio insufficient.

The mixture is evenly stirred at high speeds for about 1 to 300 seconds, and then poured into a mold or on a continuous production line operated by a conveyor belt, as an example. The mixture is blown for 0.1 to 10 minutes to be solidified as foam. The blowing and gelling process can be performed at room temperature to 200° C., preferably at 20° C. to 100° C., and further preferably 20° C. to 50° C. The foam prepared by the described methods has a non-yellowing degree of 5 under UV exposure.

Because the prepolymer of the embodiment is obtained by reacting the diisocyanate trimer and the diol, the problems experienced in the conventional process, such as low reaction rate of the aliphatic isocyanate prepolymer and low gelling rate of the foam having high mechanical strength are improved. Moreover, the heating process of the foam of the embodiment is less costly and less time consuming than conventional foams. The foam of the embodiment has non-yellowing index of 5. The foam of the embodiment has a blowing time of 1 to 300 seconds, preferably 5 to 100 seconds, and further preferably of 10 to 50 seconds. The foam of the embodiment has a gelling time of 0.1 to 10 minutes, preferably 0.5 to 5 minutes, and further preferably of 0.5 to 2 minutes. The gelling reaction of the foam does not need to have a high temperature heating process, and it can be performed at room temperature to 200° C., preferably at 20° C. to 100° C., and further preferably at 20° C. to 50° C. Because the prepolymer of the embodiment has high reaction rate, the process is more simplified, compared to the related art.

In addition to being blown to form the foam, the prepolymer of the embodiment may be directly reacted with diol and/or triol to form an unbubbled product such as a film by gelling.

### EXAMPLES

### Example 1

404 g of HDI trimer (N3600, commercially available from Bayer) was heated to 70° C., and then added 400 g of PPG (PPG-1000, commercially available from Bayer) to react to form a prepolymer, as shown in Formula 5. 172 parts by weight of the prepolymer, 4 parts by weight of water, 0.5 parts by weight of pentane served as blowing agent, and 3 parts by weight of surfactant (L580, commercially available from Union Carbide, U.S.A) were completely mixed for 20 seconds and then poured into an open mold. After foaming and gelling for 1 minute, a foam product having good mechanical strength, a non-yellowing index of 5, and a density of 40 kg/cm³ was obtained.

### Example 2

A mixture of 168 g of HDI (Desmodur H, commercially available from Bayer) and 504 g of HDI trimer (N3600, commercially available from Bayer) were heated to 70° C., and then slowly added 1000 g of PPG (PPG-1000, commercially available from Bayer) dropwise to react to form a prepolymer, as shown in Formula 7. 172 parts by weight of the prepolymer, 4 parts by weight of water, 1 part by weight of pentane served as blowing agent, and 3 parts by weight of surfactant (L580, commercially available from Union Carbide, U.S.A) were completely mixed for 20 seconds and then poured into an open mold. After foaming and gelling for 1 minute, a foam product having good mechanical strength, a non-yellowing index of 5, and a density of 40 kg/cm³ was obtained.

### Example 3

495 g of HDI (Desmodur H, commercially available from Bayer) was added 800 g of PPG (PPG-1000, commercially available from Bayer), reacted at a temperature of 100° C. for a period less than 1 minute, and then cooled to 45° C. The resulting mixture was then added 45 g of HDI trimer (N3600, commercially available from Bayer), and then stirred for 30 minutes to obtain the prepolymer product having 2 to 4 isocyanate functional groups, as shown in Formula 7.

The described prepolymer was blown as below. 55 g of triol (1103, molecule weight is about 3000, commercially available from Bayer), 4 g of surfactant (L580, commercially available from Union Carbide, U.S.A), and 0.9 g of tin catalyst (T9, commercially available from ECHO chemical) were evenly mixed, added to 159 g of the prepolymer, stirred for 10 seconds, and then poured into a mold to blow. The foaming ratio of the prepolymer reaches 12 times as a measuring result.

The described prepolymer was adopted to prepare a film as below. 6 g of the prepolymer, 25 g of triol (1103, Mw was about 3000, commercially available from Bayer), and 0.9 g of tin catalyst (T9, commercially available from ECHO chemical) were charged in a mold, thoroughly mixed by stirring, and then put into a vacuum oven until bubbles therein completely disappeared. The degassed mixture was reacted at a temperature of 80° C. for 2 hours and 120° C. for 4 hours to cure and obtain a film (20 cm*5 cm*0.3 cm). The film properties such as tensile strength (kg/cm²), extension ratio (%), and 100% tensile strength (kg/cm²) were measured and tabulated in Table 1.

### Comparative Example 1

495 g of HDI (Desmodur H, commercially available from Bayer), 25 g of HDI trimer (N3600, commercially available from Bayer), and 800 g of PPG (PPG-1000, commercially available from Bayer) were directly mixed to form a mixture A.

The mixture A was blown as below. 55 g of triol (1103, Mw was about 3000, commercially available from Bayer), 4 g of surfactant (L580, commercially available from Union Carbide, U.S.A), and 0.9 g of tin catalyst (T9, commercially available from ECHO chemical) were evenly mixed, added to 159 g of the mixture A, stirred for 10 seconds, and then poured into a mold to blow. The foaming ratio of the mixture A of Comparative Example 1 was poor, and the product further collapsed as a measuring result. The prepolymer of the disclosed embodiment had better foaming effect than the mixture A as shown in the blowing comparison between Example 3 and Comparative Example 1. Because the triol was added in the blowing process and the multi-isocyanate prepolymer had a similar molecular weight with the triol, good reactivity therebetween occurred. Meanwhile, the trimer and HDI had much smaller molecule weight than the triol, such that the properties of the product in Comparative Example 1 were reduced due to uneven reaction therebetween.

The described mixture A was adopted to prepare a film as below. 6 g of the mixture A, 25 g of triol (1103, Mw was about 3000, commercially available from Bayer), and 0.9 g of tin catalyst (T9, commercially available from ECHO chemical) were charged in a mold, thoroughly mixed by stirring, and then put into a vacuum oven until bubbles therein completely disappeared. The degassed mixture was reacted at a temperature of 80° C. for 2 hours and 120° C. for 4 hours to cure and obtain a film (20 cm*5 cm*0.3 cm). The film properties such as tensile strength (kg/cm²), extension ratio (%), and 100% tensile strength (kg/cm²) were measured and tabulated in Table 1.

TABLE 1

| | | | 100% tensile |
|---|---|---|---|
| | | tensile strength | strength |
| | extension ratio | (kg/cm²) | (kg/cm²) |
| Example 3 | 85% | 15.6 | 12.5 |
| Comparative Example 1 | 23% | 6.5 | 5.5 |

As shown in Table 1, even if the prepolymer and the mixture A had similar composition ratios, the film prepared from the prepolymer of the disclosed embodiment still had better physical properties than the film prepared from the mixture A. Because the triol was added in the blowing process and the multi-isocyanate prepolymer had a similar molecular weight with the triol, good reactivity therebetween occurred. Meanwhile, the trimer and HDI had much smaller molecule weight than the triol, such that the properties of the product in Comparative Example 1 were reduced due to uneven reaction therebetween.

### Example 4

178 g of HDI (Desmodur H, commercially available from Bayer) was added 80 g of PPG (PPG-1000, commercially available from Bayer), and reacted at a temperature of 100° C. until the terminal hydroxyl groups of the PPG being transferred to isocyanate groups, such that a diisocyanate prepolymer was obtained. Subsequently, 40 g of triol (KH570, Mw was about 300, commercially available from Takeda, Japan) was added to the diisocyanate prepolymer, cooled to 45° C., and then stirred for 30 minutes to obtain the prepolymer product having 3 diisocyanate functional groups, as shown in Formula 9.

The described prepolymer was blown as below. 55 g of triol (1103, Mw was about 3000, commercially available from Bayer), 4 g of surfactant (L580, commercially available from Union Carbide, U.S.A), and 0.9 g of tin catalyst (T9, commercially available from ECHO chemical) were evenly mixed, added to 159 g of the prepolymer, stirred for 10 seconds, and then poured into a mold to blow. The foaming ratio of the prepolymer reaches 10 times as a measuring result.

The described prepolymer was adopted to prepare a film as below. 6 g of the prepolymer, 25 g of triol (1103, Mw was about 3000, commercially available from Bayer), and 0.9 g of tin catalyst (T9, commercially available from ECHO chemical) were charged into a mold, thoroughly mixed, and then put into a vacuum oven until bubbles therein completely disappeared. The degassed mixture was reacted at a temperature of 80° C. for 2 hours and 120° C. for 2 hours to cure and obtain a film (20 cm*5 cm*0.3 cm). The film properties such as tensile strength (kg/cm²), extension ratio (%), and 100% tensile strength (kg/cm²) were measured and tabulated in Table 2.

### Comparative Example 2

178 g of HDI (Desmodur H, commercially available from Bayer), 80 g of PPG (PPG-1000, commercially available from Bayer), and 40 g of triol (KH570, Mw was about 300, commercially available from Takeda, Japan) were directly mixed to form a mixture B.

The mixture B was blown as below. 55 g of triol (1103, Mw was about 3000, commercially available from Bayer), 4 g of surfactant (L580, commercially available from Union Carbide, U.S.A), and 0.9 g of tin catalyst (T9, commercially available from ECHO chemical) were evenly mixed, added to 159 g of the mixture B, stirred for 10 seconds, and then poured into a mold to blow. The foaming ratio of the mixture B of Comparative Example 2 was poor, and the product further collapsed as a measuring result. The prepolymer of the disclosed embodiment had better foaming effect than the mixture B as seen by the blowing comparison between Example 4 and Comparative Example 2. Because the triol was added in the blowing process and the multi-isocyanate prepolymer had a similar molecular weight with the triol, good reactivity therebetween occurred. Meanwhile, the trimer and HDI had much smaller molecule weight than the triol, such that the properties of the product in Comparative Example 2 were poor due to uneven reaction therebetween.

The described mixture B was adopted to prepare a film as below. 6 g of the mixture B, 25 g of triol (1103, Mw was about 3000, commercially available from Bayer), and 0.9 g of tin catalyst (T9, commercially available from ECHO chemical) were charged into a mold, thoroughly mixed, and then put into a vacuum oven until bubbles therein completely disappeared. The degassed mixture was reacted at a temperature of 80° C. for 2 hours and 120° C. for 4 hours to cure and obtain a film (20 cm*5 cm*0.3 cm). The film properties such as tensile strength (kg/cm²), extension ratio (%), and 100% tensile strength (kg/cm²) were measured and tabulated in Table 1.

TABLE 2

| | | | 100% tensile |
|---|---|---|---|
| | | tensile strength | strength |
| | extension ratio | (kg/cm²) | (kg/cm²) |
| Example 4 | 37% | 6.93 | 6.9 |
| Comparative Example 2 | 10% | 2.25 | 1.8 |

As shown in Table 1, even if the prepolymer and the mixture B had similar composition ratios, the film prepared from the prepolymer of the disclosed embodiment still had better physical properties than the film prepared from the mixture B. Because the triol was added in the blowing process and the multi-isocyanate prepolymer had a similar molecular weight with the triol, good reaction rate therebetween occurred. Meanwhile, the trimer and HDI had much smaller sizes than the triol, such that the properties of the product in Comparative Example 2 were poor due to uneven reaction therebetween.

While the invention has been described by way of example and in terms of the disclosed embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A multi-isocyanate prepolymer obtained by reacting a diisocyanate trimer with a diol, wherein the diisocyanate trimer and the diol have a molar ratio from 2:1 to 4:1.

2. The multi-isocyanate prepolymer as claimed in claim 1, wherein the diisocyanate trimer comprises isophorone diisocyanate trimer, hexamethylene diisocyanate trimer, methylene-bis(4-cyclohexylisocyanate) trimer, 4,4′-dicyclohexylmethane diisocyanate trimer, or combinations thereof.

3. The multi-isocyanate prepolymer as claimed in claim 1, wherein the diol comprises polyester polyol or polyether polyol, the diol has terminal hydroxyl groups, and the molecule weight of the diol are from 200 to 3000.

4. The multi-isocyanate prepolymer as claimed in claim 1 formed further by reacting with a diisocyanate.

5. The multi-isocyanate prepolymer as claimed in claim 4, wherein the diisocyanate comprises isophorone diisocyanate, hexamethylene diisocyanate, Methylene-bis(4-cyclohexylisocyanate), 4,4′-dicyclohexylmethane diisocyanate, or combinations thereof.

6. The multi-isocyanate prepolymer as claimed in claim 1 applied as a foam formula, wherein the foam formula further comprises surfactant, catalyst, blowing agent, diol or triol, and/or water.

7. The multi-isocyanate prepolymer as claimed in claim 1 applied as an unbubbled material formula, wherein the unbubbled material formula further comprises diol or triol.

8. A multi-isocyanate prepolymer obtained by reacting a diisocyanate prepolymer with a triol, wherein:
the diisocyanate prepolymer and the triol have a molar ratio from 3:1 to 6:1; and
the diisocyanate prepolymer is obtained by reacting a diisocyanate with a diol, wherein the diisocyanate and the diol have a molar ratio from 2:1 to 4:1.

9. The multi-isocyanate prepolymer as claimed in claim 8, wherein the diisocyanate comprises isophorone diisocyanate, hexamethylene diisocyanate, Methylene-bis(4-cyclohexylisocyanate), 4,4′-dicyclohexylmethane diisocyanate, or combinations thereof.

10. The multi-isocyanate prepolymer as claimed in claim 8, wherein the diol or triol comprises polyester polyol or polyether polyol, the diol or triol has terminal hydroxyl group, and the molecule weight of diol are from 200 to 3000.

11. The multi-isocyanate prepolymer as claimed in claim 8 applied as a foam formula, wherein the foam formula further comprises surfactant, catalyst, blowing agent, diol or triol, and/or water.

12. The multi-isocyanate prepolymer as claimed in claim 8 applied as an unbubbled material formula, wherein the unbubbled material formula further comprises diol or triol.
